(19) United States
(12) Patent Application Publication
(10) Pub. No.: US 2003/0113437 A1
(43) Pub. Date: Jun. 19, 2003

(54) Metered application of imageable media

(76) Inventors: Fernando Lopes, Richmond (CA); Jonathan William Goodin, Tsawwassen (CA); Lesley M. Otsig, Burnaby (CA)

Correspondence Address: OYEN, WIGGS, GREEN & MUTALA, 480 - THE STATION, 601 WEST CORDOVA STREET, VANCOUVER, BC, V6B 1G1, CA

(21) Appl. No.: 10/050,607

(22) Filed: Jan. 18, 2002

Related U.S. Application Data
(60) Provisional application No. 60/339,803, filed on Dec. 17, 2001.

(51) Int. Cl. A61L 2/00

(52) U.S. Cl. 427/2.13

(57) ABSTRACT

A printing plate is prepared by applying an imageable medium to a lithographic substrate by positive-displacement metering of the imageable medium. The method is applied in on-press platemaking machines, separate platemaking machines and plate coating machines.

This application claims the benefit of U.S. Provisional Application No. 60/339,803, filed Dec. 17, 2001.

### BACKGROUND OF THE INVENTION

Lithographic printing presses use a printing master such as a printing plate that is mounted on a cylinder of the printing press. The master carries a lithographic image on its surface and a print is obtained by applying ink to said image and then transferring the ink from the master onto a receiver material, which typically is paper. In conventional lithographic printing, ink as well as an aqueous fountain solution (also called dampening liquid) are supplied to the lithographic image which consists of oleophilic (or hydrophobic, i.e. ink-accepting, water-repelling) areas as well as hydrophilic (or oleophobic, i.e. water-accepting, ink-repelling) areas. In so-called driographic printing, the lithographic image consists of ink-accepting and ink-adhesive areas and during this kind of printing, only ink is supplied to the master.

Printing masters have traditionally been obtained by the computer-to-film method, wherein various pre-press steps such as typeface selection, scanning, color separation, screening, trapping, layout and imposition are accomplished digitally and each color selection is transferred to graphic arts film using an image-setter. After processing, the film can be used as a mask for the exposure of an imaging material on a plate, also known as a plate precursor. After the plate has been processed, a printing plate is obtained which can be used as a master.

More recently the computer-to-plate (CTP) method has gained a lot of interest. This method, also called direct-to-plate method, bypasses the creation of film because the digital document is transferred directly to a plate precursor by means of a plate-setter machine. In the field of such computer-to-plate methods the following improvements are at present being studied:

(i) On-press imaging.

A special type of a computer-to-plate process involves the exposure of a plate precursor while it is mounted on a plate cylinder of a printing press. This is done by means of an image-setter that is integrated in the press. This method may be called ‘computer-to-press’ and printing presses with an integrated plate-setter are sometimes called digital presses. A review of digital presses is given in the Proceedings of the Imaging Science & Technology's 1997 International Conference on Digital Printing Technologies (Non-impact Printing 13). Computer-to-press methods have been widely described and are well known to those schooled in the art of commercial printing. Typical plate materials used in computer-to-press methods are based on ablation. A problem associated with ablative plates is the generation of debris, which is difficult to remove and may disturb the printing process or may contaminate the exposure optics of the integrated image-setter. Other methods require wet processing with chemicals. Such processes may damage or contaminate the electronics and optics of the integrated image-setter and other devices of the press.

(ii) On-press coating.

Whereas a plate precursor normally consists of a sheet-like support and one or more functional coatings, computer-to-press methods have been described wherein a composition, capable of forming a lithographic surface upon image-wise exposure and optional processing, is provided directly on the surface of a plate cylinder of the press. Techniques have also been described in which a coating of a hydrophobic layer is applied directly on the hydrophilic surface of a plate cylinder. After removal of the non-printing areas by ablation, a master is obtained. However, ablation should be avoided in computer-to-press methods, as discussed above. In U.S. Pat. No. 5,713,287 a computer-to-press method is described wherein an imageable medium is applied directly on the surface of a plate cylinder. The imageable medium is converted from a first water-sensitive or oil-sensitive property to an opposite water-sensitive or oil-sensitive property by image-wise exposure.

(iii) Thermal imaging.

Most of the computer-to-press methods referred to above use so-called thermal or heat-mode materials, i.e. plate precursors or on-press coatable compositions, which comprise a compound that converts absorbed light into heat. The heat which is generated on image-wise exposure triggers a (physico) chemical process, such as ablation, polymerization, insolubilization by cross-linking of a polymer, decomposition, or particle coagulation of a thermoplastic polymer latex, and after optional processing, a lithographic image is obtained.

(iv) The development of special functional coatings

Another major trend in plate-making is special functional coatings. Examples of such coatings include ones that require no wet processing, or that may be processed with plain water, ink or fountain solution. Such materials are especially desirable in computer-to-press methods so as to avoid damage or contamination of the optics and electronics of the integrated image-setter by contact with the processing liquids. Most such plates are, however, ablative, and have a multi-layer structure that makes them less suitable for on-press coating. However, non-ablative plates that can be processed with plain water have been described. Such plates also allow on-press processing, either by wiping the exposed plate with water while it is mounted on the press, or by the ink or fountain solution applied during the first runs of the printing job.

(v) On-site plate coating

Preparation of printing masters by coating and imaging on-press was mentioned above in section (ii) as was reusing the master substrate by means of removing or cleaning the printing surface from the substrate. Coating of plate masters off-press has existed since the 1960's as hand-wiped plates. This process, due to poor coating quality associated with hand coating, has fallen out of favour given increased demand for quality printing and has in general been replaced by precoated plates. In the case of hand-coated plates, however, the substrates were not reused. There is value in reusing the lithographic substrate as the materials and production of such substrate can be costly. This is becomes even more feasible for shorter print runs where the mechanical properties of the substrate do not degrade significantly. There is thus interest in the process of reusing lithographic substrates by removing the printing master from the press, and installing it in a separate device whereby the printing surface is removed, the substrate is recoated, and optionally imaged for reuse in printing.

A computer-to-press method has also been disclosed in which an oleophilic substance is image-wise transferred from a foil to a rotary press cylinder by melting said substance locally with a laser beam. The strip-shaped transfer foil has a narrow width compared to the cylinder and is translated along a path which is parallel to the axis of the cylinder while being held in close contact with the surface of the cylinder so as to build up a complete image on that surface gradually. As a result, this system is rather slow and requires a long downtime of the printing press, thereby reducing its productivity.

An on-press coating method has been described wherein an aqueous liquid, comprising a hydrophilic binder, a compound capable of converting light to heat and hydrophobic thermoplastic polymer particles, is coated on the plate cylinder so as to form a uniform, continuous layer thereon. Upon image-wise exposure, areas of the coated layer are converted into a hydrophobic phase, thereby defining the printing areas of the printing master. The press run can be started immediately after exposure without any additional treatment because the layer is processed by interaction with the fountain and ink that are supplied to the cylinder during the press run. So the wet chemical processing of these materials is ‘hidden’ to the user and accomplished during the first runs of the printing press. After the press run, the coating can be removed from the plate cylinder by an on-press cleaning step. Such methods of on-press coating, on-press exposure and on-press cleaning of the master are commercially attractive because, contrary to conventional lithographic printing, they can be carried out without specialized training or experience. Such presses require less human intervention than conventional presses.

As may be seen from the foregoing, the technology of on-press imaging and on-site platemaking has made major strides and represents a major benefit to industry. However, a problem associated with coating substrate materials, both on-press and in dedicated off-press coating and imaging equipment, is that the method produces an insufficient coating quality, characterized by a low consistency and a high frequency of coating artifacts.

It is an objective of the present invention to provide a method by which the coating of imageable media onto a lithographic surface mounted on a press or in an off-press platemaking device may be undertaken in a controlled and accurate manner, making possible the commercial application of the fully integrated on-press coating, imaging and printing process in addition to the reuse of substrates both on and off press.

### BRIEF SUMMARY OF THE INVENTION

A printing plate is prepared by applying an imageable medium to a lithographic substrate by positive-displacement metering of the imageable medium. The method is applied in on-press platemaking machines, separate platemaking machines and plate coating machines.

### BRIEF DESCRIPTION OF THE DRAWING

FIG. 1 shows a machine that embodies the present invention in a fully on-press implementation

### DETAILED DESCRIPTION OF THE PREFERRED EMBODIMENT

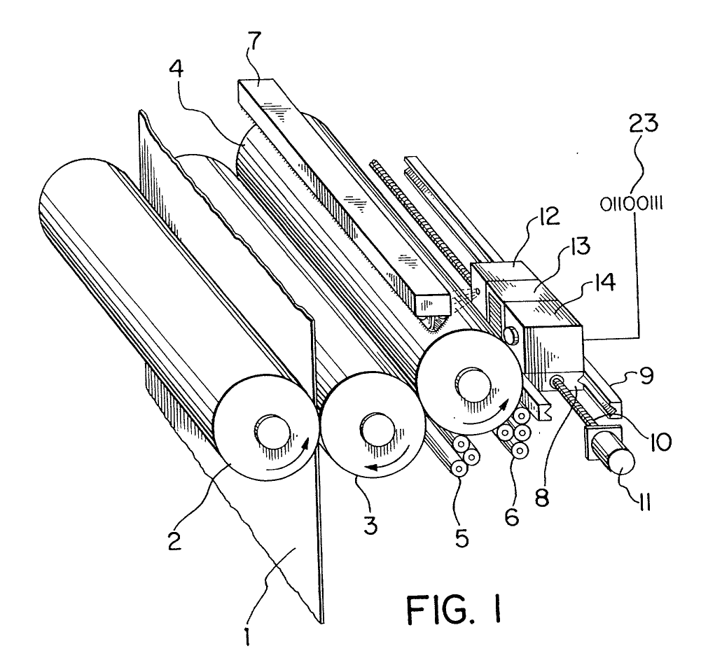

Referring to FIG. 1, which represents the printing section of a lithographic press, paper 1 (either in sheet or web form) is compressed between impression cylinder 2 and blanket cylinder 3. Blanket cylinder 3 is in contact with image cylinder 4, which replaces the plate cylinder in a conventional press and forms the lithographic base for the purposes of executing the invention. In the present application for letters patent, the term “lithographic base” is used to describe any substrate onto which an imageable coating will be applied to make a lithographic element. Suitable examples of lithographic bases are anodized aluminum, chrome, nickel, steel, and ceramics such as alumina (Al₂ O₃) and zirconia (ZrO₂). Zirconia is particularly desirable as it is very durable, hydrophilic and refractory while having a low thermal conductivity. Low thermal conductivity minimizes the amount of laser energy required to heat up the coating of imageable material to change its response to water or oil.

The material of the lithographic base may be chosen to be optimal for a particular kind of imageable material or medium to be coated upon it. For bi-level media, chromium, stainless steel, hydrophilic ceramics, glasses, nickel, anodized aluminum, grained aluminum, and hydrophilic polymer such as treated polyester, are good choices. For mono-level so-called switchable polymers there is a wide choice of lithographic base materials such as aluminum, polyester, treated paper, and steel, because the functioning of the lithographic process for such materials is not based on the behavior of the lithographic base with respect to water, oil or ink. In these cases, the main consideration is mechanical durability and adhesion for the coating material.

In a first preferred embodiment of the present invention, imaging cylinder 4 is a seamless cylinder, thus being able to run faster and with less vibration compared to a plate cylinder having an elongated gap along the length of the image cylinder (not shown) for clamping the plate. The image cylinder 4 is being inked by a water/ink system using fountain solution rolls 5 and ink rolls 6. Rolls 5 and 6 will be merged in some inking systems known as “integrated” inking chain. Alternatively, the press can operate in waterless offset (also known as “dry offset”) mode in which fountain solution rolls 5 are not used.

Up to this point the press is largely conventional and well known. A cleaning unit 7 is mounted near image cylinder 4 and is capable of washing off most of the ink, water and imaged layer used on a previous print run. The cleaning unit is similar to the well known “blanket washer” units employed in modern presses to clean the blanket cylinder between print runs, with the exception that extra solvents may have to be added to dissolve most of the imaged layer. Additional cleaning units can be used to clean blanket cylinder 3 and other cylinders in accordance with modern press design.

A linear track 9 is rigidly mounted parallel to image cylinder 4. A traveling carriage 8 is traversing image cylinder 4 under the control of motor 11 and lead screw 10. The motion of image cylinder 4 and motor 11 are synchronized using shaft encoders in a manner similar to all drum imaging devices. Drum imaging devices are well known and have been commercially available for many years.

Thus, no further details of the synchronization and handling of the image data will be given.

A coating unit 12, optional curing unit 13 and imaging unit 14 are mounted on carriage 8 and capable of traversing the image cylinder 4. Coating unit 12 coats a recording media in liquid form onto the imaging cylinder 4, after the imaging cylinder 4 has been cleaned. In the preferred embodiment of the present invention, the coating technique is spraying. A variety of alternative application techniques exist and may be used in the present invention. For example, the recording media can be applied by a roller, a sponge, brush, blade, or cloth wetted with the coating fluid where either contact is made directly with the imaging cylinder 4, or the liquid coating bridges the gap between the applicator and the imaging cylinder 4.

The coating thickness must be very carefully controlled so as to be uniform and close to the target coating thickness. If the coating is thicker or thinner than desired, it can have negative impact on the imaging and printing performance of the media. Thinner coatings typically have lower durability than thicker coatings. Controlling the thickness is critical to ensuring consistent performance of the media and as a result the flow of liquid recording media to the coating unit on the printing press or on the off-press device must be very carefully controlled. Additionally, some coating applications require that multiple liquid components be mixed together in precise ratios prior to coating. Controlling the coating thickness job-wise also facilitates the use of thinner coatings when lower durability is acceptable in order to reduce consumption of coating material, for example, when the printing job's run length is shorter.

As shown in FIG. 1, imageable material metering unit 15 is installed inline with the coating unit 12 and serves to draw coating liquid from a reservoir or reservoirs (not shown) and supply the fluid to the coating unit. The metering unit may beneficially be located very close to or be integral with the coating unit such that fluid line volumes are minimized to reduce amount of imageable material wasted during flushing of the fluid lines. Additionally, the reduced elasticity of shorter fluid lines may be beneficial for achieving a sharp response when the coating unit starts or stops quickly. Optionally, when multiple coating materials must be mixed prior to being supplied to the coating unit, multiple metering units, in an alternative embodiment of the present invention, can draw from separate reservoirs and supply the materials to a mixing unit prior to being supplied to the coating unit. Imageable material metering unit 15 consists of a positive displacement device. Examples of such devices include gear, piston, diaphragm, or peristaltic pumps. Benefits of such devices include some inherent restriction to backflow without any additional check valves. The reservoir(s) may consist of containers and or hoses, or fluid paths that contain the media components.

Methods of metering liquids on-press or in off-press platemaking devices are known. Typically, these methods involve supplying a driving pressure for the liquid, for example with a centrifugal pump, and then imposing a restrictive element to control the flow. The restrictive element is typically a small orifice or a valve. These methods are not practical for the precise metering of recording media because the flowing properties of liquids through small orifices or valves is sensitive to variations in viscosity. Orifices and valves also cannot be easily adjusted should the desired liquid flow rate change on the press or on the off-press coating unit. If the driving pressure used is high, then the flow can be controlled by means of a very small orifice or valve opening but these configurations pose a greater risk of plugging and manufacturing tolerances must be very small. If a lower driving pressure is used, then a larger orifice or valve opening may be used but in this case the system is then very susceptible to changes in the driving pressure or changes in the fluid pathway that might impose a variable restriction.

With the use of positive displacement metering units, the above limitations are avoided. In a positive-displacement metering unit, a volume containing liquid is altered so as to force liquid in or out of the volume. By controlling the dimensions and the rate of change of the volume, the liquid flowing in and/or out of the volume can be metered. The size of the fluid displacing parts can be made extremely small and thus can accurately meter fluids at a much lower flow rate and supply pressure with much less risk of plugging. Examples of suitable positive displacement metering units include gear pumps, piston pumps, and peristaltic pumps.

In the present application for letters patent, the term “curing” is used to describe any process by which a substantially liquid medium is brought to a substantially solid condition and specifically includes, but is not limited to, polymeric cross-linking and drying. The curing is accelerated by heat, either radiant or hot air, generated by optional curing unit 13. Use of U.V. light to accelerate curing is also possible. The thickness of the cured coating layer is from 0.2 to 10 microns typically, thus the amount of material to be cured is small.

After curing, the coating is imaged by a multi-channel laser head 14 according to the pre-press data files 23. In order to image the surface of coated imaging cylinder 4 in a short time (in the order of several minutes or less) a large number of beams are required as well as a relatively high power. Multi-beam laser imagers are well known. By the way of example, a laser array is described in U.S. Pat. No. 4,743,091, which is incorporated herein by reference. The number of beams required depends on the required imaging time, power, and the maximum rotational speed of the image cylinder 4.

Preferably, the reaction of the imageable media to the incident laser illumination from multi-channel laser head 14 is purely thermal, so that any type of laser can be used. Laser diodes operating in the near infra-red are the preferred source. Typically the cylinder is imaged at a resolution of 2400 DPI, but the invention is not limited to this resolution. For conventional printing the coatings tested required between 0.1 J/cm² to 0.5 J/cm² while for waterless printing, energy requirements were 0.4 J/cm² to 0.8 J/cm². When a laser supplying 20 W is used on an 8 page press (80 cm×100 cm signature size) the time required to expose the image varies from (80 cm×100 cm×0.1 J/cm²): 20 W=40 sec for the best case, up to (80 cm×100 cm×0.8 J/cm²): 20 W=320 sec for the worst case, using the lowest sensitivity waterless printing polymer. Thus, these times can only be shortened by having a more powerful laser head and typically are not limited by the data rate of the pre-press system.

The illumination from multi-channel laser head 14 modifies the response of the coating to water and to oil. Through the choice of thermally imageable medium, this may be achieved by changing the actual oleophobicity or hydrophobicity of the coating material itself. Alternatively, it may be achieved by removing the layer to expose an underlying layer, which may be the hydrophilic base material of the plate, or of image cylinder 4 in the case where the coating was done directly onto image cylinder 4. Removal of areas of the layer may be by ablation, or, more preferably, by the use of a thermally imageable medium based on coalescing polymer particles. Where illuminated, the polymer particles coalesce to form hydrophobic areas that are ink-loving. Where the material is not illuminated, it may be removed by the fountain process described below, thereby producing the printing master.

To print, the image cylinder is engaged with the blanket cylinder and the inking system. Fountain solution roll 5 applies fountain solution (water) to the plate, where it wets the hydrophilic areas. This is followed by ink rolls 6 applying ink (preferably oil based) to the hydrophobic areas.

The present invention is not limited to coatings applied directly to a seamless cylinder of a press. A separate lithographic base may be applied to the printing cylinder of a press and the process may be conducted on that lithographic base.

The present invention is further not limited in any way to on-press applications. It may just as effectively be applied to a separate platemaking machine or platesetter. In such an embodiment of the present invention, there would obviously be no printing or post-printing cleaning step executed on the platesetter, as the purpose of a platesetter is to produce a printing plate. In such an embodiment, the completed plate would be removed from the platesetter and then placed on a press for the actual printing. Again it would be a generalized lithographic base that is used as the surface to be coated by the method of the invention. In such an embodiment, the steps in manufacturing a plate would include, but not be limited to, cleaning the lithographic base, coating an imageable material, metering the flow of imageable material, optionally curing the layer so coated and optionally imaging the layer. The required apparatus for such an embodiment shares most of the aspects of that shown in FIG.1. However, in a purely platemaking machine, the paper 1, cylinders 2 and 3, blanket washer 7 and rollers 5 and 6 of the machine shown in FIG. 1 are not required.

In the present application for letters patent, the term “imaging machine” is used to describe any member of the family of machines that are used to image printing plates. This specifically includes, but is not limited to, plate-setters and presses.

The invention may also be applied to machines that coat plates with imageable media without imaging the media. One embodiment of this aspect of the invention is based on the machine shown in FIG.1, but with the imaging and printing facilities removed, leaving the coating and optional curing facilities to describe this embodiment. In particular, the invention may be applied to re-usable lithographic base. In the present application for letters patent, the term re-usable lithographic base is used to describe any substrate onto which an imageable coating can be applied to make a lithographic element, and which, after use, may have its lithographic coatings removed to restore it to its original pre-coating condition with its associated lithographic properties.

Any one of a number of different thermally imageable materials may be employed to as thermally imageable coating material. In the preferred embodiment of the present invention, the negative-working thermally imageable materials described by commonly-owned U.S. patent application Ser. Nos. 09/745548, 09/909792, 09/909964, and 09/785339, as well as those of U.S. Pat. No. 3,476,937 and U.S. Pat. No. 6,001,536, are preferred. All of the thermally imageable materials disclosed in these applications and patents are developable using aqueous media. The specifications of U.S. patent application Ser. Nos. 09/745548, 09/909792, 09/909964, and 09/785339 are hereby incorporated in full by reference. All of the media presented in those applications comprise hydrophobic polymer particles, a material that absorbs the light from a laser and one or more of an inorganic salt, a metal complex, an organic base and an organic acid.

U.S. Pat. No. 3,476,937 Vrancken describes a material that is thermally imageable and which is composed either of finely divided particles of a hydrophobic thermoplastic polymer arranged in discrete contiguous relationship, or consisting essentially of a dispersed phase of such polymer particles distributed generally homogeneously through a continuous phase of a hydrophilic binding agent applied from an aqueous medium. The heat applied is sufficient to at least partially coalesce the polymer particles in the affected areas of a layer of the material and to significantly reduce the fluid permeability of the layer in these affected areas. The layer may contain other materials such as colorants or color developable agents.

U.S. Pat. No. 6,001,536 Vermeersch describes the use of a thermally imageable material comprising hydrophobic thermoplastic polymer particles dispersed in a non-hardened hydrophilic binder and a compound capable of converting light to heat. The hydrophobic thermoplastic particles have a glass transition temperature T_{g} of at least 80° C. Upon exposure to light that is convertible by the light to heat converting compound, the thermoplastic particles in the illuminated portions of the thermally imageable material coalesce.

In a subsequent development step, the unexposed areas of the thermally imageable material may be removed by plain tap water or an aqueous liquid. In the particular patent the hydrophilic binder is selected from the group consisting of poly(meth)acrylic acid, poly(meth)acrylamide, polyhydroxyethyl(meth)-acrylate and polyvinylmethyl-ether.

The materials described in all six of these patent applications and patents are all imageable by laser heads as described in the preferred embodiment of the present invention and may all be dried using hot air or radiant heat. They are all insensitive to room light and therefore do not require special lighting conditions for their processing.

Alternative negative working thermally imageable materials may be employed to create thermally imageable layer 2. Some examples of these are described in U.S. Pat. No. 5,491,046 (De Boer), U.S. Pat. No. 5,641,608 (Grunwald), U.S. Pat. No. 5,925,497 (Li), U.S. Pat. No. 6,124,425 (Nguyen) U.S. Pat. No. 6,242,155 (Yamasaki) and WO9739894 (Parsons).

U.S. Pat. No. 5,491,046 De Boer describes a thermally imageable material that was developed for lithographic printing, and that comprises an admixture of a resole resin, a novolac resin, a latent Bronsted acid and an infrared absorber. This material may be employed as a negative-acting medium by heating it in an additional step with intense infrared radiation from curing unit 8 after imaging with multichannel laser head 9. By employing an alkaline developer, the unexposed areas of thermally imageable layer 2 may be removed to produce a gravure etch mask. Clearly, because of the use of corrosive alkaline developer, this embodiment of the invention is not ideal for implementation on an integrated apparatus, and is better implemented in an alternative embodiment where the development of the mask is carried out in a separate developing unit.

U.S. Pat. No. 5,641,608 Grunwald describes a thermally imageable resist, developed for printed circuit board application, and comprising a styrene-maleic-anhydride copolymer. Various examples of this invention are described using either organic solvents or alkaline solutions as developers. This material is also preferably employed in systems where the mask development is separated from the preceding steps of the method.

U.S. Pat. No. 5,925,497 Li describes a negative-working photosensitive composition containing a polymer of the formula B(X)(Y), wherein B represents an organic backbone, each X independently is an acidic group or salt thereof and each Y independently is a photo-curable group and a photo-initiating compound or compounds with sensitivity up to 850 nm. Areas of this material struck by light of wavelength matching the absorption spectrum photo-cure and thereby become insoluble in aqueous and organic media. The areas not irradiated with that light remain soluble in the fountain. U.S. Pat. No. 5,928,833 Matthews describes a radiation-sensitive coating that includes

a) core-shell particles, the core-shell particles comprising an oleophilic water-insoluble, heat-softenable core component (A) having a minimum film-forming temperature above room temperature and a shell component (B) which is soluble or swellable in aqueous medium, the shell component (B) being a polymer containing carboxylic acid, sulphonic acid, sulphonamide, quaternary ammonium, or amino groups; and,

b) a radiation-sensitive component (C) which, on exposure to radiation, changes the solubility characteristics of the coating, wherein the core (A) and the shell(B) components of the particles remain as separate components prior to the application of heat to the coating, but coalesce on the application of heat to the coating, and wherein the core-shell particles are distributed throughout the radiation-sensitive component (C),

wherein the radiation-sensitive component (C) does not comprise part of the core-shell particles.

U.S. Pat. No. 6,124,425 Nguyen describes a near infrared absorption polymer comprising

a) a near infrared absorption segment, which exhibits strong absorption bands between 780 and 1200 nm;

b) a processing segment providing film forming properties and solubility in aqueous solutions having pH between 2.0 and 14.0;

c) a thermally reactive segment, which undergoes localized chemical or physical reactions, with or without catalysts, upon localized exposure to near infrared laser light so that said polymer becomes locally insoluble in aqueous solutions, the polymer being soluble in aqueous solutions prior to exposure to near infrared light.

U.S. Pat. No. 6,242,155 Yamasaki describes a family of photopolymer compositions for recording images by exposure to infrared beams. The composition comprises a photothermal converter and a polymer that is thermally decarboxylated. Examples are given of the use of these photopolymers in making lithographic plates. While no separate development step was employed, the lithography process did include treatment with either plain tap water or a fountain solution mix of water, IPA, triethylamine and HCI to remove unexposed portions of the plate. WO9739894 (Parsons) describes, coated in particular on a lithographic base, a complex of a developer-insoluble phenolic resin and a compound which forms a thermally frangible complex with the phenolic resin. This complex is less soluble in the developer solution than the uncomplexed phenolic resin. However when this complex is imagewise heated the complex breaks down so allowing the noncomplexed phenolic resin to the dissolved in the developing solution. Thus the solubility differential between the heated areas of the phenolic resin and the unheated areas is increased when the phenolic resin is complexed. Preferably a laser-radiation-absorbing material is also present on the lithographic base. A large number of compounds which form a thermally frangible complex with the phenolic resin have been located. Examples of such compounds are quinolinium compounds, benzothiazolium compounds, pyridinium compounds and imidazoline compounds

All of the thermally imageable materials, as described in U.S. Pat. No. 5,491,046 (De Boer), U.S. Pat. No. 5,641,608 (Grunwald), U.S. Pat. No. 5,925,497 (Li), U.S. Pat. No. 6,124,425 (Nguyen) U.S. Pat. No. 6,242,155 (Yamasaki) and WO9739894 (Parsons), may be employed in their respective ways in negative working methods and may be used to prepare gravure etch masks by the method of the present invention. To the extent that they employ developers that are to a lesser or greater degree corrosive or dangerous, they are preferably executed on apparatus that separate the mask development from the preceding steps of coating, drying and imaging.

The present invention is not limited to thermally imageable media, and it may be very effectively employed with other imageable media responding to various wavelengths of light from alternative lasers and light sources. Thermally imageable materials are, nevertheless, preferred due to the relaxed ambient conditions required compared with media that is daylight sensitive.

The present invention is also not limited to negative working imageable media and positive working media may be employed, as described, for example, by Gelbart in U.S. Pat. No. 5,713,287, which is hereby incorporated herein by reference.

Example of orifice/valve metering:

1. Thermally imageable media is prepared as an emulsion by mixing 6 g UCAR 471, 12 g 5 wt % sodium carbonate in deionized water, 12 g 1 wt % ADS 830A in ethanol, into 36 g deionized water.

2. Liquid thermally imageable media at a temperature of 5 degrees Celsius is pumped through a loop of tubing to a tee fitting. At the tee fitting, a return tube carries fluid back to the pump inlet. A third tube connects the tee fitting to the inlet of a control valve with restricted diameter. The valve feeds liquid media to the spray gun mounted on the press. When the valve is opened, positive pressure generated by the pump feeds the liquid media through the spray gun.

3. A grained, anodized aluminum sheet is clamped onto the press cylinder.

4. With the control valve open and the gun spraying, the coating unit is translated along the length of the press cylinder while the cylinder is rotating and in this sense, the substrate is coated with thermally imageable media to create a coating thickness of approximately 0.5 microns.

5. The coated substrate is subsequently measured by gravimetric means to have a coating thickness of 0.5 microns.

6. Process steps 1 to 4 are repeated but with liquid recording media at a temperature of 40 degrees Celsius whereby the coating fluid viscosity is much lower.

7. The coated substrate is subsequently measured by gravimetric means to have a coating thickness of 0.6 microns.

8. This variation in coating thickness between samples is attributable to the sensitivity of orifice type metering devices to changes in fluid viscosity.

Example of positive displacement metering:

1. Thermally imageable media is prepared as an emulsion by mixing 6 g UCAR 471, 12 g 5 wt % sodium carbonate in deionized water, 12 g 1 wt % ADS 830A in ethanol, into 36 g deionized water.

2. Liquid recording media at a temperature of 5 degrees Celsius is drawn from a small reservoir into a small gear pump driven by a motor at constant speed. The gear pump feeds the liquid recording media to a spray gun mounted on the press.

3. A grained, anodized aluminum sheet is clamped onto the press cylinder

4. With the gear pump rotating and the gun spraying, the coating unit is translated along the length of the press cylinder while the cylinder is rotating and in this sense, the substrate is coated with thermally imageable media to create a coating thickness of approximately 0.5 microns.

5. The coated substrate is subsequently measured by gravimetric means to have a coating thickness of 0.5 microns.

6. Process steps 1-4 are repeated but with liquid recording media at a temperature of 40 Celsius.

7. The coated substrate is subsequently measured by gravimetric means to have a coating thickness of 0.5 microns.

In the above examples the chemical supplies were sourced as follows:

Sodium carbonate from Aldrich Chemicals Milwaukee, Wis., U.S.A.

UCAR 471 polymer from Union Carbide, Danbury, Conn., U.S.A.

ADS

830A an infra-red absorbing dye from American Dye Source Inc. Montreal, Quebec, Canada.

Grained, anodized aluminum was obtained from Precision Lithoplate of South Hadley, Mass.

There have thus been outlined the important features of the invention in order that it may be better understood, and in order that the present contribution to the art may be better appreciated. Those skilled in the art will appreciate that the conception on which this disclosure is based may readily be utilized as a basis for the design of other methods and apparatus for carrying out the several purposes of the invention. It is most important, therefore, that this disclosure be regarded as including such equivalent methods and apparatus as do not depart from the spirit and scope of the invention.

What is claimed is

1. A method for applying a coating of thermally imageable material onto a lithographic base, said method comprising positive displacement metering of said thermally imageable material while said thermally imageable material is being applied to said lithographic base.

2. The method of claim 1, wherein said thermally imageable material is negative-working thermally imageable material.

3. A method for applying a coating of negative-working thermally imageable material onto a lithographic base, said method comprising positive displacement metering of said negative-working thermally imageable material while said negative-working thermally imageable material is being applied to said lithographic base, said negative-working thermally imageable material comprising hydrophobic polymer particles, a material that absorbs the light from said laser and one or more of an inorganic salt, a metal complex, an organic base and an organic acid.

4. A method for applying a coating of thermally imageable material to a re-usable lithographic base, said method comprising metering of said thermally imageable material while said thermally imageable material is being applied to said re-usable lithographic base.

5. The method of claim 4, wherein said thermally imageable material is negative-working thermally imageable material.

6. The method of claim 4, wherein said thermally imageable material comprises hydrophobic polymer particles, a material that absorbs the light from said laser and one or more of an inorganic salt, a metal complex, an organic base and an organic acid.

7. A method for applying a coating of imageable material to a lithographic base, said method comprising
a. mounting said lithographic base on an imaging machine and
b. metering said imageable material while said imageable material is being applied to said lithographic base.

8. A method for applying a coating of thermally imageable material to a lithographic base, said method comprising
a. mounting said lithographic base on an imaging machine and
b. positive displacement metering of said thermally imageable material while said thermally imageable material is being applied to said lithographic base.

9. The method of claim 8, wherein said thermally imageable material is a negative-working thermally imageable material.

10. A method for applying a coating of thermally imageable material to a hydrophilic lithographic base, said method comprising
a. Mounting said hydrophilic lithographic base on a imaging machine and
b. positive-displacement metering of said thermally imageable material while said thermally imageable material is being applied to said hydrophilic lithographic base,
said thermally imageable material comprising hydrophobic thermoplastic polymer particles dispersed in a non-hardened hydrophilic binder and a compound capable of converting light to heat.

11. A method for making a thermally imageable printing plate, said method comprising
a. mounting a hydrophilic lithographic base on an imaging machine,
b. preparing a thermally imageable medium, said preparing comprising making an emulsion of hydrophobic polymer particles, a material that absorbs the light from said laser and one or more of an inorganic salt, a metal complex, an organic base and an organic acid.
c. positive-displacement metering said imageable material while said thermally imageable material is being applied to said hydrophilic lithographic base.

12. A method according to claim 11 further including the step of varying the rate of metering job-wise.

13. An apparatus for applying a coating of imageable material to a lithographic base, said apparatus comprising
a. a positive displacement metering unit disposed to meter said imageable material,
b. an imageable material applicator disposed to apply a coating of said imageable material onto said lithographic base.

14. The apparatus of claim 13, additionally comprising an imaging head disposed to image said coating.

15. The apparatus of claim 13, wherein said imageable material is thermally imageable material.

16. The apparatus of claim 13, wherein said imageable material is negative-working thermally imageable material.

17. The apparatus of claim 14, wherein said imageable material is thermally imageable material.

18. The apparatus of claim 14, wherein said imageable material is negative-working thermally imageable material.
